(19) United States
(12) Patent Application Publication
(10) Pub. No.: US 2023/0070504 A1
(43) Pub. Date: Mar. 9, 2023

(54) HANDS-FREE DEVICES AND METHODS FOR THE PREVENTION AND TREATMENT OF MIGRAINES AND OTHER HEAD AND NECK DISORDERS

(71) Applicant: Jason Michael Parnes, New City, NY (US)

(72) Inventor: Jason Michael Parnes, New City, NY (US)

(21) Appl. No.: 17/822,967

(22) Filed: Aug. 29, 2022

Related U.S. Application Data
(60) Provisional application No. 63/238,832, filed on Aug. 31, 2021.

(51) Int. Cl. A61F 7/12; A61F 7/00

(52) U.S. Cl. CPC A61F 7/12; A61F 7/0085; A61F 7/007; A61F 2007/0017; A61F 2007/0056; A61F 2007/0075; A61F 2007/0094

(57) ABSTRACT

A hands-free intraoral device for imparting a chilling treatment to a zone of intraoral tenderness within an oral cavity of a user. The hands-free intraoral device includes a carrier having a proximal end and a distal end. The distal end of the carrier is configured to be received within the oral cavity of the user and the proximal end of the carrier is configured to extend to a front and/or externally of the oral cavity of the user. A cooling element formed of a thermally conductive material selected to impart the chilling treatment to the zone of intraoral tenderness. A positioning element is operable to urge at least a portion of the cooling element in abutment with the zone of intraoral tenderness, providing for a hands-free positioning of the cooling element in abutment with the zone of intraoral tenderness.

### CROSS-REFERENCE TO RELATED APPLICATION

This application claims the benefit of priority of U.S. provisional application No. 63/238,832 filed Aug. 31, 2021, the contents of which are herein incorporated by reference.

### BACKGROUND OF THE INVENTION

The present invention relates to intraoral devices, and more particularly to intraoral devices which cool an area of a plexus formed by one or more of a posterior superior or a middle superior alveolar branch of an ipsilateral maxillary nerve, known herein as the zone of intraoral tenderness.

A tension-type headache, migraine, post-traumatic headache, atypical facial pain, cervical muscle hyperactivity, and other conditions are each symptoms of ipsilateral intraoral tenderness, as discovered by Dr. Mark H. Friedman, DDS. As disclosed in U.S. Pat. No. 5,527,351, issued Jun. 18, 1996, U.S. Pat. No. 5,676,691, issued Oct. 14, 1997, and U.S. Patent Application Publication Number U.S 2007/0032547, published Feb. 8, 2007, Dr. Friedman prevented numerous patients from ever experiencing a migraine and other ailments again, including the Inventor herein, by chilling the zone of intraoral tenderness for forty minute sessions via metal probes applied to the zone and cooling the temperature of the probes to a range of between 0° C.-10° C.

Despite 39 million Americans suffering from the debilitating symptoms of a migraine alone, no invention has been proposed to better chill the zone of intraoral tenderness than the original prototype and its similarly hard to utilize successors, which are collectively over twenty years old. Since Dr. Friedman's passing over a decade ago, no significant resources have been put into addressing or improving his apparatus or his treatment methodology despite their clinical success. Because his devices required a practitioner to properly position and a patient to hold the probes and the number of practitioners utilizing his methods remain limited, there is a need for an improved intraoral device for the treatment of migraines, headaches, and other head and neck disorders and conditions.

Research institutions, pharmaceutical and digital health companies, and charities have been set up to counteract migraines. Despite the billions of dollars in resources invested in the space, no improvements have been made on his clinically successful methodology.

Further, the user's application of Dr. Friedman's probes naturally led users to stick them as far back in the intraoral cavity as possible, due to the shape of the probe and location of the zone. Positioning errors remained due to the user manually holding the probes in position. To withstand the 40 minutes of holding the probes in position, users are encouraged to place a pillow under each arm to steady their arms and hold their hands in position. Then, wrap cloth around the medical tubes as a handle to tolerate the cold. Still, the free-wielding probes led to cooling the wrong area of the intraoral cavity and holding the probes in place for 40 minutes led to muscle fatigue. The difficulty in correctly applying the device and that a dentist needed to apply the treatment led to its lack of public acceptance along with a rise in the popularity of opioids at the time of discovery and far less frequent social media usage by the public.

Further, headache and migraine symptoms specifically are often misdiagnosed by doctors as a possible tumor leading to a costly and potentially dangerous CT Scan or MRI. If no tumor is present, treatment often includes medications, rest, TENs devices, and the avoidance of causal factors including but not limited to certain foods, work, the sun or light, and sound. The pain leaves many with only the hope of prayer because of the debilitating nature of a migraine and lack of reliable treatments available.

Migraine treatment is a specifically crowded space. Current options have a low efficacy rate, are primarily pharmaceutical solutions, and are temporary, meaning at some point in time the user will get another migraine. Despite early success in treating and preventing migraines and other ailments by chilling the zone of intraoral tenderness, none of the current devices or methodologies provide assurance in knowing the hard to reach zone is cooled with a high enough consistency to ensure effective treatment.

It is therefore an object of the present invention to provide more effective treatment solutions for migraines and other ailments mentioned above by chilling the zone of intraoral tenderness.

More particularly, it is an object of the present invention to cool the zone of intraoral tenderness by providing a hands-free device that is more easily applied, yet still more reliably positions the cooling element in abutment with the zone of intraoral tenderness, as further described herein.

Furthermore, it is an object of the current invention that the cooling medium can come in many forms to better cool the hard to access zone of intraoral tenderness.

It is another object of the current invention to provide for more approximate sizing by relying on external factors including but not limited to standing height or jaw size, or a custom mold model allowing users to more reliably point the cooling element towards the zone of intraoral tenderness.

It is another object of the current invention to provide a cooling element extending, protruding, or attached to a device, carrier, clip, or any general widget, allowing users to more reliably direct the cold towards the zone of intraoral tenderness.

### SUMMARY OF THE INVENTION

In one aspect of the present invention, a hands-free intraoral device for imparting a chilling to an area of a plexus formed by one or more of a posterior superior or a middle superior alveolar branch of an ipsilateral maxillary nerve, known herein as the zone of intraoral tenderness, within an oral cavity of a user is disclosed. The hands-free intraoral device includes a carrier having a proximal end and a distal end. The distal end is configured to be received within the back of the oral cavity and the proximal end is configured to extend to a front and/or external of the oral cavity. A cooling element is disposed at the distal end of the carrier. The cooling element is formed of a thermally conductive material selected to impart the chilling to the zone of intraoral tenderness. A conduit communicates a cooling fluid through the hands-free device to the cooling element. A positioning element to position at least a portion of the cooling element in abutment with the zone of intraoral tenderness. The positioning element provides for a hands-free positioning of the cooling element in abutment with the zone of intraoral tenderness.

In other aspects of the invention, a hands-free intraoral device for imparting a heat transfer from the zone of intraoral tenderness within an oral cavity of a user is disclosed. The hands-free intraoral device includes a carrier having a proximal end and a distal end, the distal end is configured to be received within the oral cavity and the proximal end is configured to extend to a front and/or externally from the oral cavity. A cooling element is disposed at the distal end of the carrier. The cooling element is formed of a thermally conductive material selected to receive the heat transfer from the zone of intraoral tenderness to the cooling element. A heat exchanger is configured to transfer the heat from the cooling element. A positioning element to position at least a portion of the cooling element in abutment with the zone of intraoral tenderness. The positioning element provides for a hands-free positioning of the cooling element in abutment with the zone of intraoral tenderness.

In some embodiments, the heat exchanger includes a thermoelectric device. The hands-free intraoral device may also include an electronics package for controlling the thermoelectric device.

In some embodiments, a communications module is configured for a wireless communication with a mobile computing device for controlling the thermoelectric cooler.

These and other features, aspects and advantages of the present invention will become better understood with reference to the following drawings, description and claims.

### BRIEF DESCRIPTION OF THE DRAWINGS

FIG. 1 is a front perspective view of a prong embodiment of the hands-free intraoral device, illustrating a left side prong as used by a user of the device.

FIG. 2 is a top plan view of the prong-embodiment.

FIG. 3 is a frontal view of a user utilizing the prong embodiment of the hands-free intraoral device shown in use for the prevention and treatment of migraines, headaches, and head and neck disorders.

FIG. 4 is a detail top front perspective view and a distal end view of the prong embodiment illustrating angular relationships of a bite pad positioning element with the probe of the hands-free intraoral device, in this case a first angular offset of 15° and a second angular offset of 0°.

FIG. 5 is a detail top front perspective view and a distal end view of the prong embodiment illustrating angular relationships of the bite pad positioning element with the probe of the hands-free intraoral device, in this case a first angular offset of 15° and a second angular offset of +10°.

FIG. 6 is a detail top front perspective view and a distal end view of the prong embodiment illustrating angular relationships of the bite pad positioning element with the probe of the hands-free intraoral device, in this case a first angular offset of 15° and a second angular offset of −10°.

FIG. 7 is a detail top front perspective view and a distal end view of the prong embodiment illustrating angular relationships of the bite pad positioning element with the probe of the hands-free intraoral device, in this case a first angular offset of 10° and a second angular offset of 0°.

FIG. 8 is a detail top front perspective view and a distal end view of the prong embodiment illustrating angular relationships of the bite pad positioning element with the probe of the hands-free intraoral device, in this case a first angular offset of 5° and a second angular offset of 0°.

FIG. 9 is a front perspective view of a user illustrating the general position of the zone of intraoral tenderness.

FIG. 10 is a front perspective view of a first U-shaped carrier embodiment of the hands-free intraoral device.

FIG. 11 is a front perspective view of a second U-shaped carrier embodiment of the hands-free intraoral device with a resilient urging positioning element.

FIG. 12 is a front perspective view of a third U-shaped carrier embodiment of the hands-free intraoral device with a first loop embodiment of the resilient urging positioning element.

FIG. 13 is a front perspective view of a fourth U-shaped carrier embodiment of the hands-free intraoral device with a flexible arm positioning element.

FIG. 14 a front perspective view of a fifth U-shaped carrier embodiment of the hands-free intraoral device with a cheek fold positioning element.

FIG. 15 is a front perspective view of a sixth U-shaped carrier embodiment of the hands-free intraoral device with a hook positioning element.

FIG. 16 is a front perspective view of a seventh U-shaped carrier embodiment of the hands-free intraoral device with a bite bar positioning element.

FIG. 17 is a front perspective view of an eighth U-shaped carrier embodiment of the hands-free intraoral device with a resilient urging positioning element, a thermoelectric cooler carried on a distal end of the U-shaped carrier, and an electronics package carried on a proximal end of the U-shaped carrier.

FIG. 18 is a front perspective view of the ninth U-shaped carrier embodiment of the hands-free intraoral device with a resilient urging positioning element, a thermoelectric cooler carried on a distal end of the U-shaped carrier, and an electronics package carried on a proximal end of the U-shaped carrier in communication with a mobile computing device.

FIG. 19 is a front perspective view of a tenth U-shaped carrier embodiment of the hands-free intraoral device with a second embodiment of a looped resilient urging positioning element, shown with an optional bite clip.

FIG. 20 is a front perspective view of an eleventh U-shaped carrier embodiment of the hands-free intraoral device with a third embodiment of a looped resilient urging positioning element, and a first lateral tensioner embodiment.

FIG. 21 is a front perspective view of a twelfth U-shaped carrier embodiment of the hands-free intraoral device with a fourth embodiment of a looped resilient urging positioning element and a second lateral tensioner embodiment.

FIG. 22 is a front perspective view of a thirteenth U-shaped carrier embodiment of the hands-free intraoral device with a looped and bite pad positioning element.

FIG. 23 is a front perspective view of the hands-free intraoral device with a with a cheek fold positioning element, with a detail view of an optional multi-lumen conduit.

FIG. 24 is a detail top perspective view of the prong embodiment illustrating a bulbous cooling element and a tooth clip positioning element.

FIG. 25 is a top perspective view of the prong embodiment illustrating a left and a right probe and the tooth clip positioning element.

FIG. 26 is a front perspective view of the prong embodiment illustrating a left and a right probe and the tooth clip positioning element, shown in use treating the zone of intraoral tenderness.

FIG. 27 illustrates a bite knob embodiment of the hands-free intraoral device.

FIG. 28 provides representative dimensions for a probe of the hands-free intraoral device.

FIG. 29 illustrates a clamping embodiment of the hands-free intraoral device.

FIG. 30 illustrates the orientation of the axes of the user.

### DETAILED DESCRIPTION

The following detailed description is of the best currently contemplated modes of carrying out exemplary embodiments of the invention. The description is not to be taken in a limiting sense but is made merely for the purpose of illustrating the general principles of the invention.

Broadly, embodiments of the present invention provide a hands-free intraoral device for imparting a chilling treatment to a zone of intraoral tenderness within an oral cavity of a user. The zone of intraoral tenderness is defined by an area of a plexus formed by one or more of a posterior superior or a middle superior alveolar branch of an ipsilateral maxillary nerve, as described in the foregoing patents and patent application publication of Dr. Mark H. Friedman, DDS.

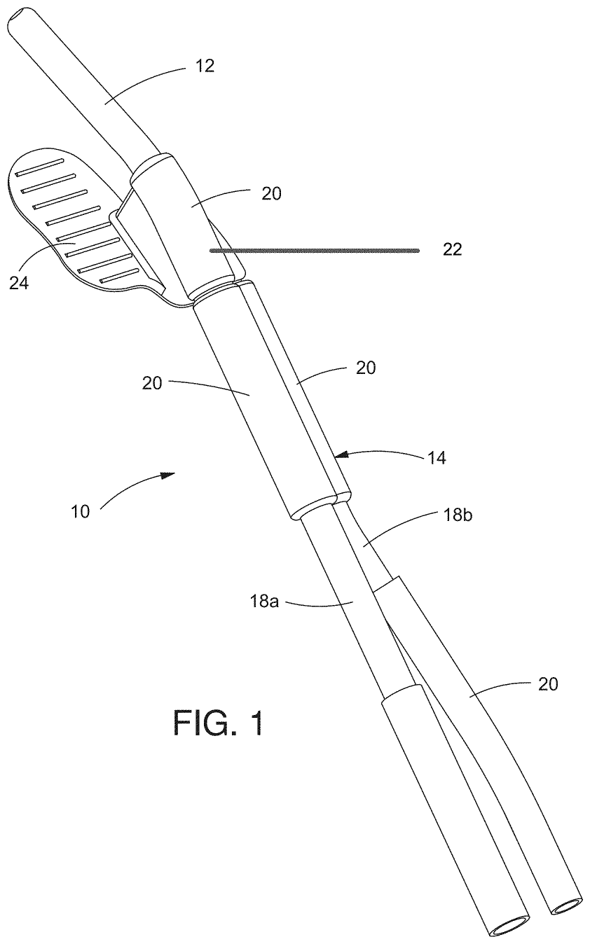

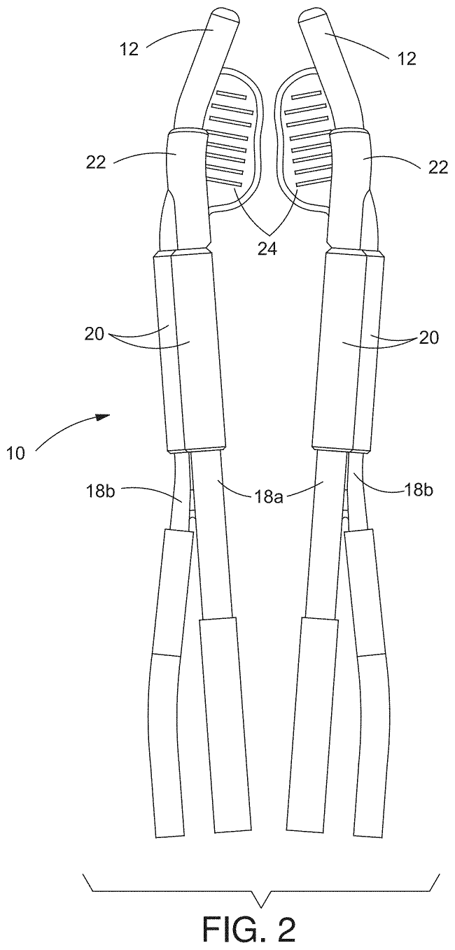

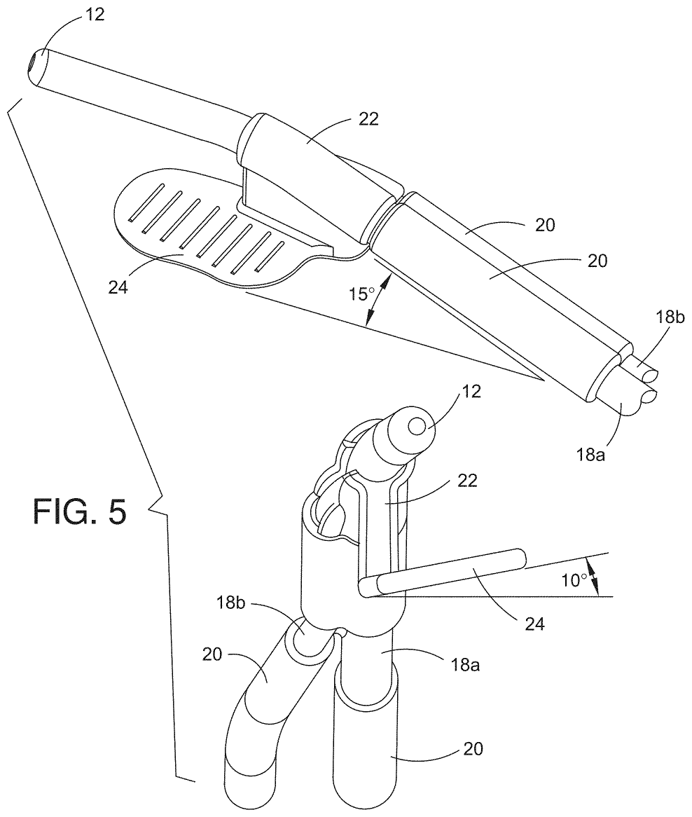

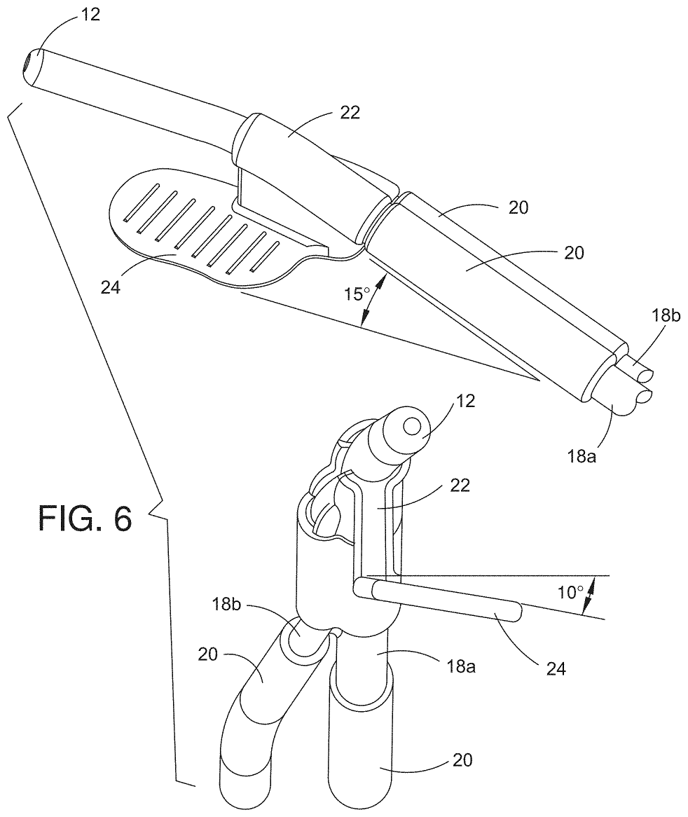

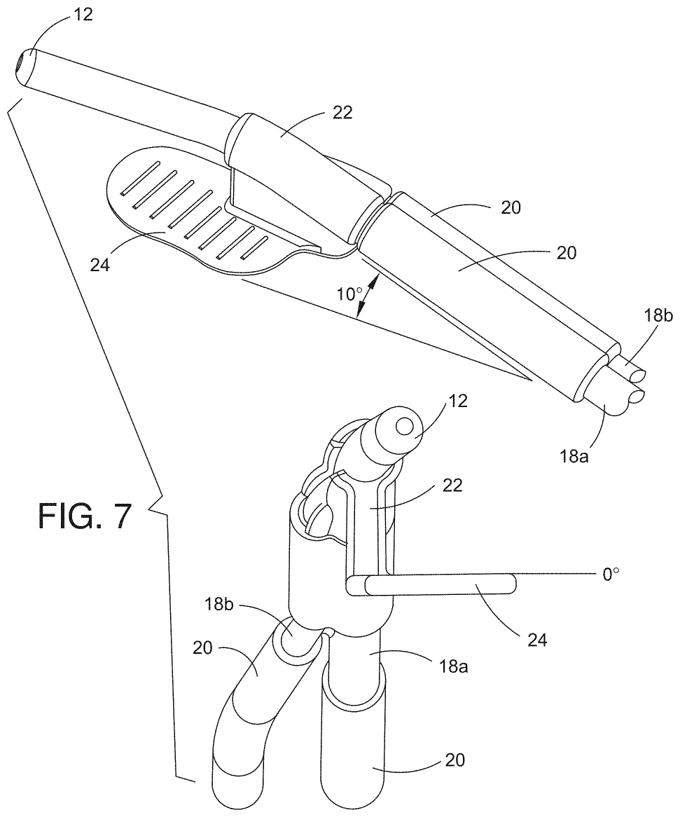

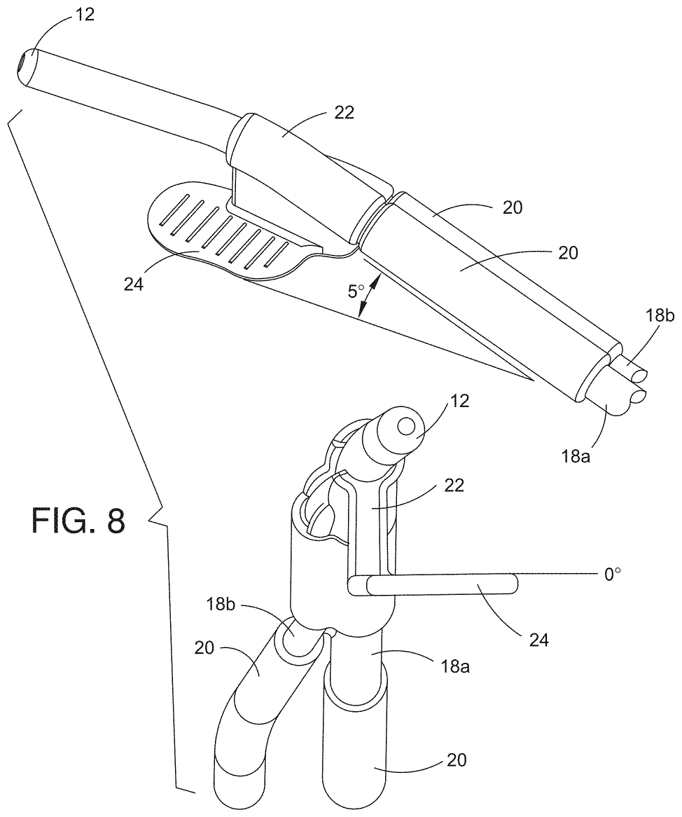

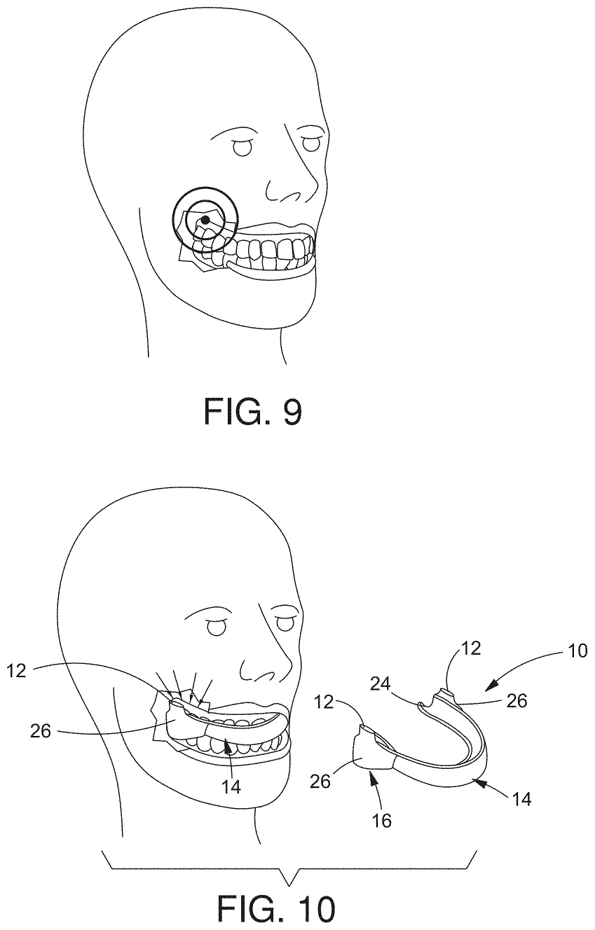

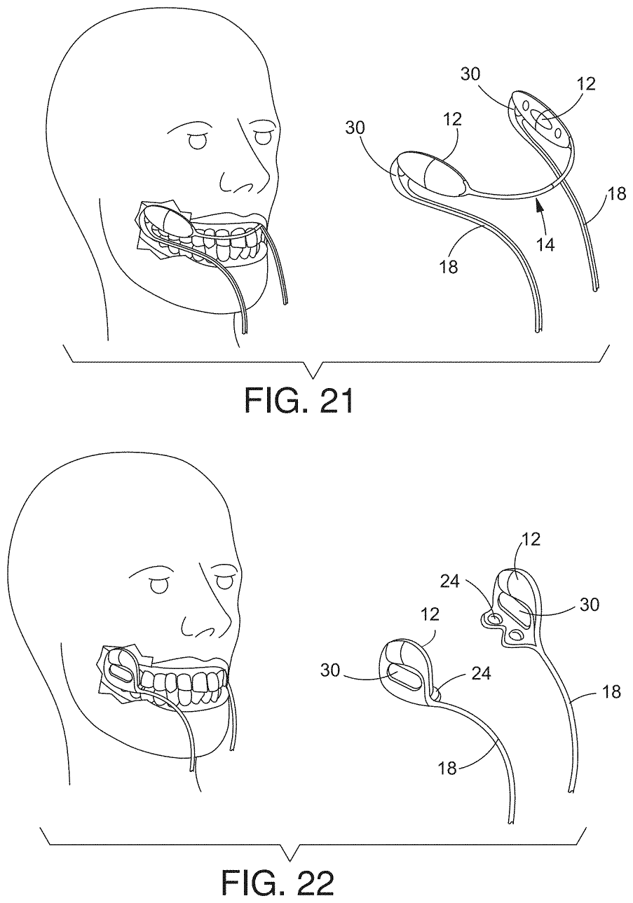

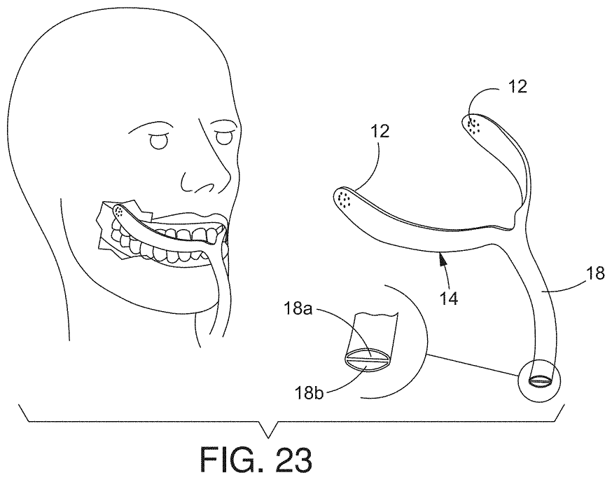

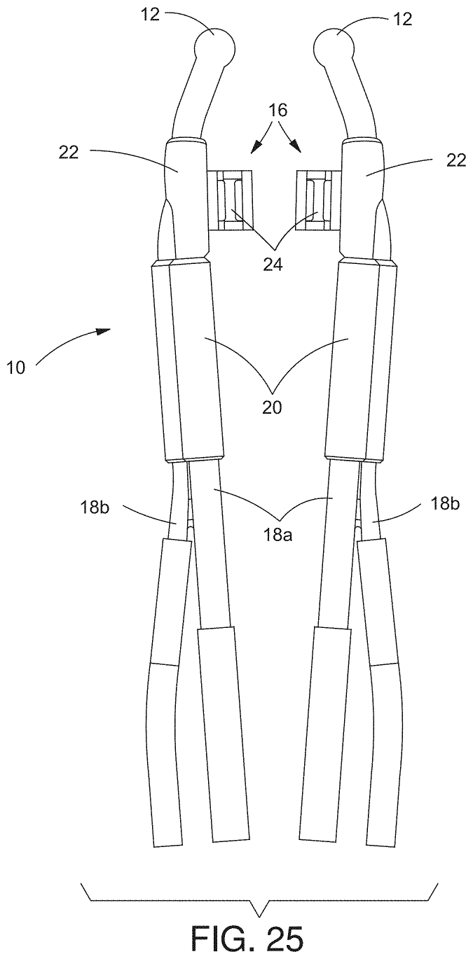

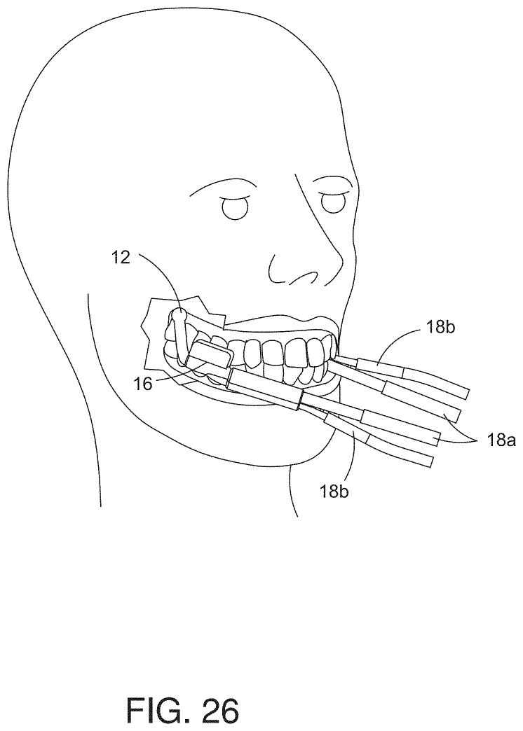

As seen in reference to the drawings of FIGS. 1-29 the hands-free intraoral device 10 includes a carrier 14 having a proximal end and a distal end. The distal end of the carrier is configured to be received within the oral cavity of the user and the proximal end of the carrier 14 is configured to extend to a front and/or external of the oral cavity of the user.

A cooling element 12 is disposed at the distal end of the carrier 14. The cooling element 12 is formed of a thermally conductive material selected to impart a chilling treatment to the zone of intraoral tenderness. In some embodiments, a cooling fluid is configured to chill the cooling element. A conduit 18 communicates a cooling fluid through the hands-free intraoral device 10 to the cooling element 12. The conduit 18 may include an inflow conduit 18a and an outflow conduit 18b for circulation of a cooling fluid through the conduit 18. The conduit 18 is in a thermally conductive contact with the cooling element 12. The thermally conductive contact may include a circulation of a cooling fluid through the cooling element 12. The conduit 18 is at least partially covered with an insulating material 20 to enhance the chilling effect at the cooling element 12 and to prevent the cooling effect from being applied to other areas of the user's oral cavity.

The cooling element 12 should have a smooth outer surface for a thermally conductive contact with the zone of intraoral tenderness and for comfort when positioned within a cheek fold at the juncture of the user's upper jaw line and cheek. The cooling element 12 may also have a bulbous, form-fitting (such as formed by a mold or a gel-like material), or other shape to enhance the thermal contact surface area of the cooling element 12 with the zone of intraoral tenderness.

Each embodiment of the hands-free intraoral device 10 includes a positioning element 16 to position at least a portion of the cooling element 12 in abutment with the zone of intraoral tenderness. The positioning element 16 provides for a hands-free positioning of the cooling element 12 in abutment with the zone of intraoral tenderness. The positioning element 16 also facilitates the retention of the hands-free intraoral device 10 in the oral cavity of the user while the user undergoes a cooling treatment session utilizing the hands-free intraoral device 10.

In some embodiments of the hands-free intraoral device 10, such as shown in FIGS. 1-8 and 24-26, the carrier 14 includes a left prong and a right prong. Each of the left prong and the right prong are dimensioned to extend the cooling element 12 into the intraoral cavity to contact the zone of intraoral tenderness.

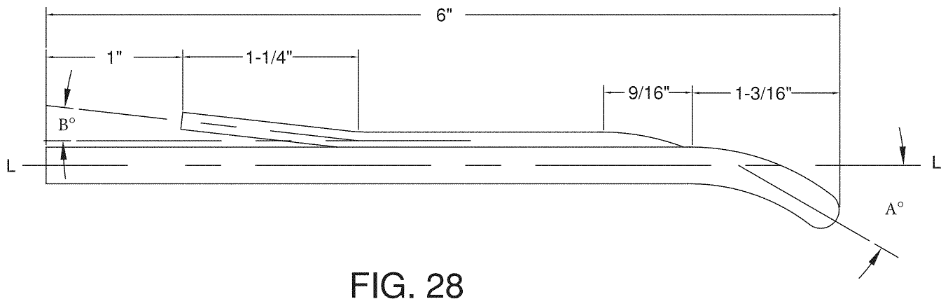

Representative dimensions of the probe are shown in FIG. 28. The cooling element 12 is offset from a longitudinal centerline of the carrier 14 by an angle A to allow the cooling element 12 to slide slightly behind the maxillary gumline, the zone of intraoral tenderness. Here, the angle A is approximately 5°. The outflow conduit may be offset by an angle of B° of approximately 6° to provide for connection of a communication tube 18c to couple with the cooling medium (not shown).

In FIGS. 1-8 and 24-26, a connector 22 is at the proximal end of each of the left prong and the right prong. The connector 22 acting as a point or part which the positioning element 16 and the cooling element 12 separately extend, forming each prong. The connector 22 may removably couple the positioning element 16 with the probes to permit replacement of the positioning element 16 for fitting of the hands-free intraoral device 10 to the user and to permit cleaning of the probes between treatments.

A bite pad 24 extends from the connector 22 along a transverse plane towards a sagittal centerline of the intraoral cavity. The bite pad 24 is configured to be operably engaged by a biting of one or more molar teeth of the user to urge the cooling element 12 in abutment with the zone of intraoral tenderness and to retain the hands-free intraoral device 10 within the intraoral cavity. The bite pads 24 and connector 22 may be formed of a rigid, a semi-rigid material, or a resilient material.

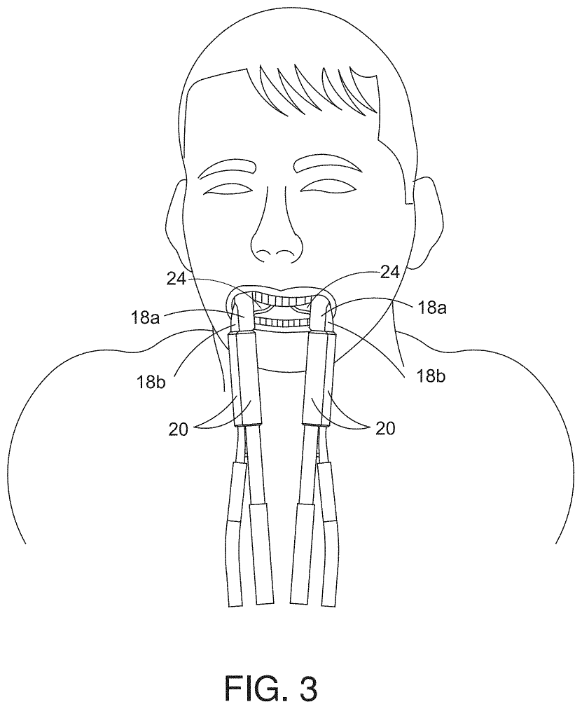

The connector 22 may also be adjustably coupled to provide a positioning adjustment such that the cooling element 12 can be positioned in abutment with the zone of intraoral tenderness. Thus, when the user engages the bite pad 24 between a selected one or more their teeth the cooling element 12 may be reliably positioned within the intraoral cavity to contact the zone of intraoral tenderness. A representative fitting of the hands-free intraoral device 10 is shown in reference to FIG. 3 where the user operably engages the bite pads 24 between their teeth.

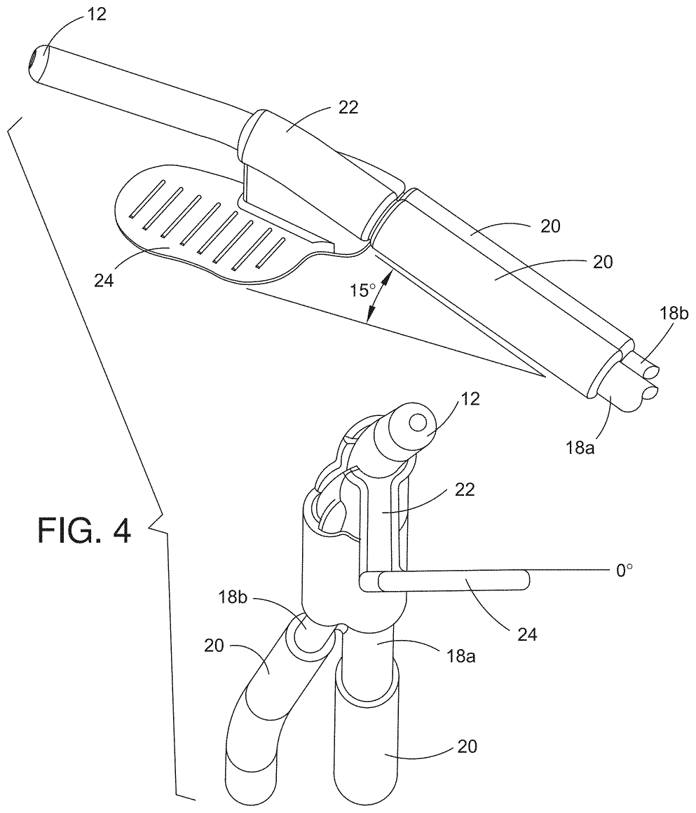

As seen in reference to FIGS. 4-8, for improved fitting of the hands-free intraoral device 10, the bite pads 24 may be oriented at one or more angular offsets so that the cooling element 12 is properly positioned in abutment with the zone of intraoral tenderness and so that the user remains comfortable with the hands-free intraoral device 10 throughout the treatment period.

The one or more angular offsets may include a first angular offset of the bite pad 24 about a frontal axis F perpendicular to the sagittal plane. The first angular offset may accommodate for a vertical elevation of the zone of intraoral tenderness from the bottom of the upper molars, by a tilting of the probes. The first angular offset is selected to elevate or lower the cooling element 12. The first angular offset may be between about 0° and 15° from the transverse plane.

The one or more angular offsets may include a second angular offset about a sagittal axis S perpendicular to the frontal plane of between about +10° and −10°. The second angular offset accommodates for variations in a lateral displacement of the zone of intraoral tenderness. The second angular offset of the bite pad 24 effectively rotates the cooling element 12 inwardly or outwardly, depending on the selected second angular offset, for proper abutment of the cooling element 12 with the zone of intraoral tenderness and so that the user remains comfortable with the hands-free intraoral device 10 throughout the treatment period. When the positioning element 16 is formed of a resilient material, the cooling element 12 may be resiliently urged in abutment with the zone of intraoral tenderness.

As seen in TABLE 1 below, a comparison of representative first angular offsets and second angular offsets (AO) of the bite pad 24 orientation are shown versus factors of comfort, secure fit, and cooling element 12 position are presented. The factors were rated on a scale of 1 to 5, with a score of 5 being the best and a score of 1 being the worst. The table illustrates that each combination was effective in positioning at least a portion of the cooling element 12 in abutment with the zone of intraoral tenderness.

TABLE 1
Comparison of Angular Offset Configurations

| Factor | 1^{st} A O15⁰ | 1^{st} AO 15⁰ | 1^{st} AO 15⁰ | 1^{st} AO 10⁰ | 1^{st} AO 5⁰ |
|---|---|---|---|---|---|
| 1 to 5 | 2^{nd} AO 0⁰ | 2^{nd} AO + 10⁰ | 2^{nd} AO − 10⁰ | 2^{nd} AO 0⁰ | 2^{nd} AO 0⁰ |
| Comfort | 3 | 4 | 4 | 2 | 1 |
| Secure Fit | 3 | 4 | 3 | 4 | 2 |
| Tip Position | 3 | 3 | 4 | 3 | 4 |
| Notes | Bite towards | Smaller bite | Probe end | Increased | Least |
| | edge of pad. | pad area. | feels further | pressure on | comfort. |
| | Drooling @ | Drooling @ 2 | from gumline, | gumline. | Most gumline |
| | 4 min. | min. | which is more | Less drooling | contact |
| | Jaw Soreness | Less bite | comfortable. | | |
| | @ 10 min. | force, stress | Less drooling | | |
| | Edge of lips | on jaw | | | |
| | itchy @ 14 min | | | | |

As seen in reference to FIGS. 10-23, the carrier 14 of many embodiments is formed as a generally U-shaped carrier. A distal end of the mouthpiece is dimensioned to be received within the intraoral cavity. In these embodiments, at least a portion of the U-shaped carrier 14 is positioned in the cheek fold, or pocket, defined between an upper jawline of the user and a cheek of the user. The cooling element 12 is disposed at a distal end of the U-shaped carrier 14.

In the example shown in reference to FIG. 10, the positioning element 16 may include a protrusion 26 defined at the distal end of the U-shaped carrier 14. The cooling element 12 is disposed at a top end of the protrusion 26 used for positioning of the cooling element 12 in abutment with the zone of intraoral tenderness. The protrusion 26 may also have a thickness that engages with a surface of the user's cheek to urge the cooling element 12 laterally for abutment with the zone of intraoral tenderness.

In the embodiment shown in reference to FIG. 10, the carrier 14 is U-shaped formed similar to that of a mouthguard. The U-shaped carrier 14 may also include a bite pad 24 extending transversely towards a sagittal centerline from an outer side of the U-shaped carrier 14. As described previously, the bite pad 24 may be oriented at one or more angular offsets to facilitate fitting of the hands-free intraoral device 10 to position the cooling element 12 in abutment with the zone of intraoral tenderness or can be molded to fit the user's mouth. The bite pad 24 is configured to be engaged by a biting of one or more molar teeth of the user teeth to position the cooling element 12 in abutment with the zone of intraoral tenderness and to retain the hands-free device within the intraoral cavity. The conduit 18 may be carried within the U-shaped carrier 14. Alternatively, a thermoelectric cooler (TEC), such as a Peltier device, or a frozen mass may be housed within the protrusion 26.

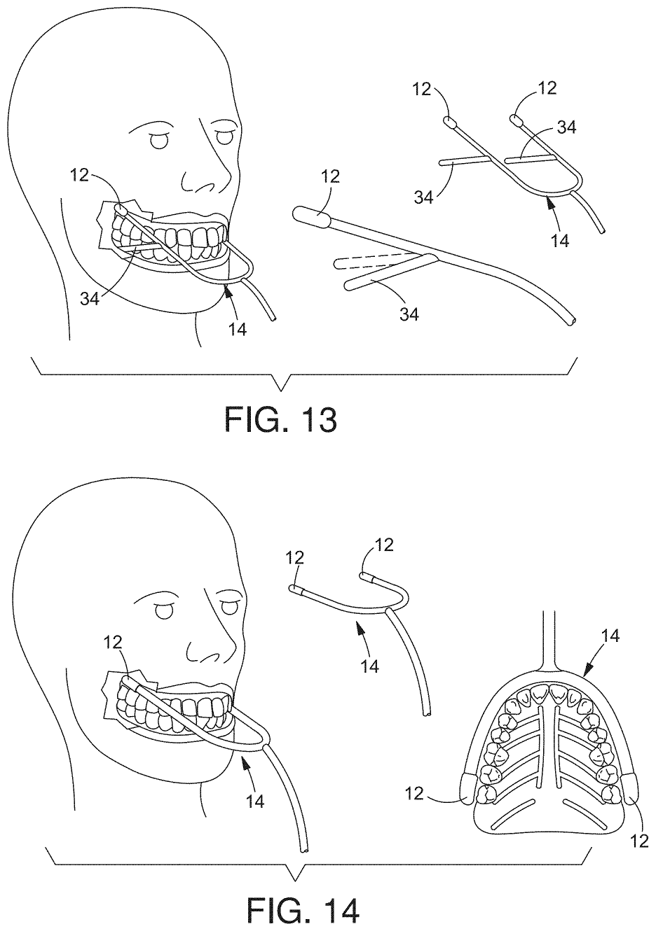

In the embodiment shown in reference to FIG. 13, at least one flexible arm 34 diverges from the carrier 14. The at least one flexible arm 34 is dimensioned to resiliently engage between the cheek fold along the jawline and the cheek of the user. The at least one flexible arm 34 is operable by the user's cheek and/or jaw to urge the cooling element 12 in abutment with the zone of intraoral tenderness, forming a multi-pronged embodiment.

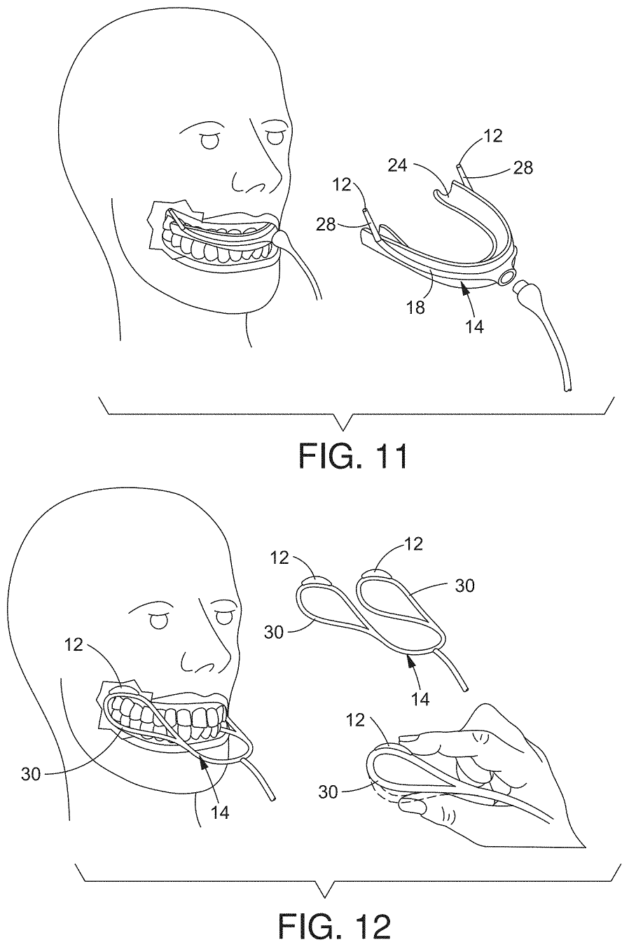

In the embodiment shown in reference to FIGS. 11, and 16-18, the positioning element 16 may be formed as a cylindrical extension 28 oriented at an elevated angular offset from the U-shaped carrier 14 about a frontal axis F, which can be a custom molded fit. The cooling element 12 is disposed at a terminal end of the cylindrical extension 28. As with previous embodiments, the bite pad 24 may be incorporated with the U-shaped carrier 14. In these embodiments, the cylindrical extension 28 may be formed of a resilient material to provide for a resilient urging of the cooling element 12 into abutment with the zone of intraoral tenderness.

In the embodiment shown in FIG. 16, a guidance plate 25 is coupled with the bite pad 24 in a vertical orientation. The guidance plate 25 facilitates positioning of the bite pad 24 and, consequently, the cooling element 12 in abutment with the zone of intraoral tenderness.

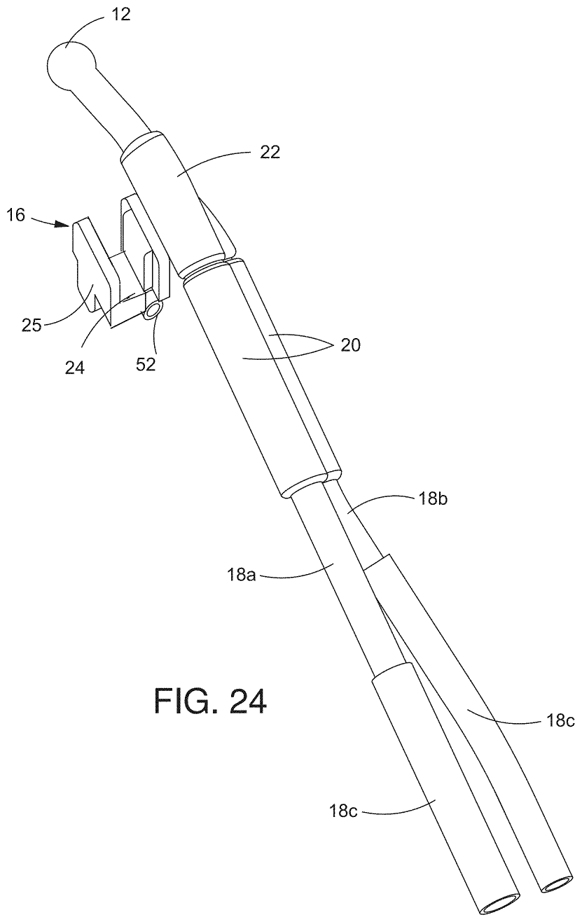

In the embodiment shown in FIGS. 24-26, the positioning element 16 is a tooth clip variation of the bite bar. In this embodiment the guidance plate 25, positioned at an interior and exterior end of the bite pad 24 are flexibly coupled via a hinge 52. The guidance plate 25 and the bite pad 24 are biased towards the carrier 14 via a tensioning mechanism (not shown) to clip the distal end of the carrier 14 to an interior and exterior surface of the one or more molars of the user.

A flexible loop 30 positioning element may be seen in reference to the drawings of FIGS. 12, and 19-22. In these embodiments, the carrier 14 may be considered a U-shaped carrier 14. The positioning element 16 is formed as a flexible loop 30 defined at the distal end of the carrier 14. The flexible loop 30 is dimensioned to be resiliently received in the cheek folds between the upper jawline, the lower jawline, and the cheeks of the user. The flexible loop 30 is operable to urge the cooling element 12 in abutment with the zone of intraoral tenderness by the user closing their lower jaw. In this case the flexible loop 30, when positioned in the cheek folds is compressed by the closure of the user's lower jaw. The cooling element 12 is disposed along an upper arc of the flexible loop 30. The flexible loop 30 may contain or be in fluid communication with the conduit 18, which may also be covered in insulation material 20. As seen in FIG. 20, a supplemental loop 31 may be provided protruding towards the user's cheek to urge the cooling element 12 inwardly towards the zone of intraoral tenderness.

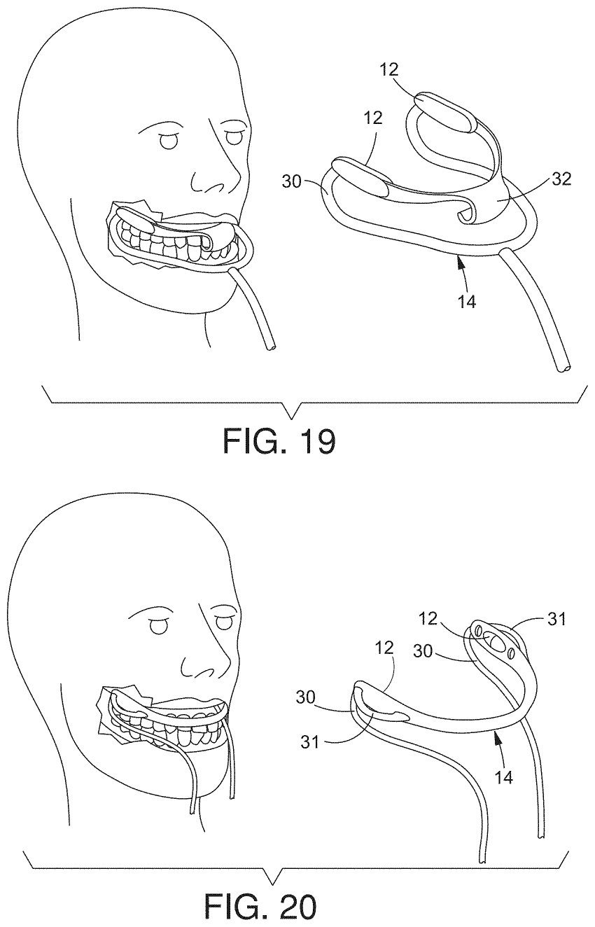

In the embodiment shown in FIG. 19 a bite clip 32 is disposed at a forward end of the flexible loop 30. The bite clip 32 is configured with a lip to engage with the user's incisors and facilitates positioning and retention of the hands-free intraoral device 10 in the intraoral cavity.

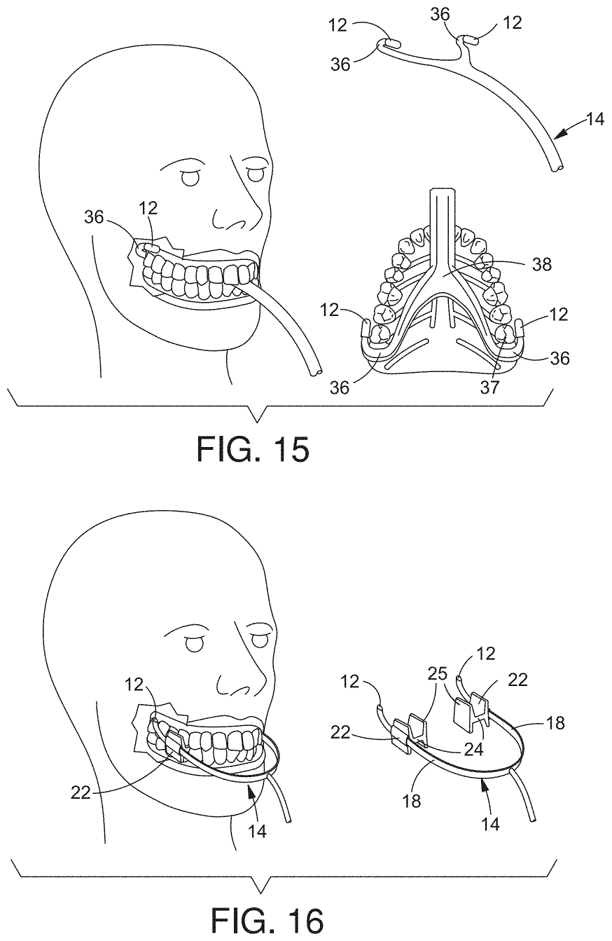

In the embodiment shown in reference to FIG. 15, the positioning element is formed as a hook 36 defined at the distal end of a wishbone shaped carrier 38. In this embodiment, the wishbone shaped carrier 38 is dimensioned to be contained or positioned in the oral pallet. The hook 36 is dimensioned for engagement around a distal end of the maxillary arch 37 of the user. The cooling element 12 is positioned at a terminal end of the hook 36 and positioned for abutment with the zone of intraoral tenderness. For sizing of this embodiment, the terminal end of the hook 36 is disposed at an elevated angle relative to a transverse plane of the intraoral cavity. The terminal end of the hook 36 may also be formed as a resilient element for resiliently urging the cooling element 12 in abutment with the zone of intraoral tenderness.

In some embodiments, a cooling medium (not shown) is in communication with the conduit 18. The cooling medium is configured to circulate the cooling fluid through the conduit 18 of hands-free intraoral device 10 and to provide the cooling of the cooling element 12.

The hands-free intraoral device 10 may also be described as imparting a heat transfer from the zone of intraoral tenderness within an oral cavity of a user. A heat exchanger 40 may be configured to transfer heat from the cooling element 12. The heat exchanger 40 may include the previously described cooling fluid circulated through the conduits 18 via the cooling medium, a thermoelectric cooler, or a thermally conductive material in contact with the vascularization of the cheek or tongue. A heat sink may be used as well and positioned under the tongue or in the upper or lower cheek fold.

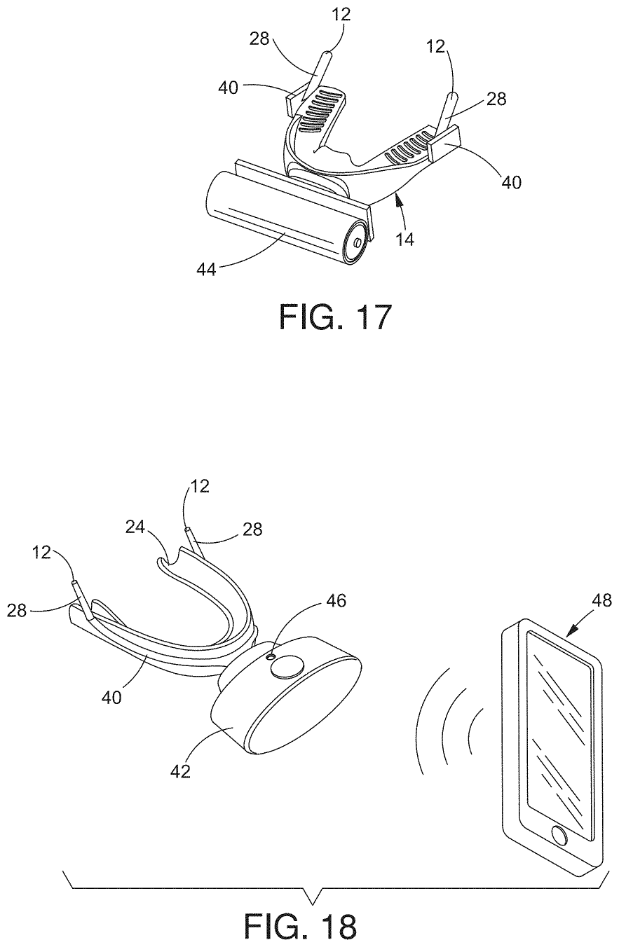

As shown in reference to FIGS. 17, 18, and 27, the heat exchanger 40 may be a component of the thermoelectric cooler thermally coupled with the cooling element 12. In this embodiment, an electronics package 42 is provided for control of the thermoelectric cooler. The electronics package 42 may include a battery 44 to power the thermoelectric cooler. In the embodiments shown, the electronics package 42 is coupled with the distal end of the carrier 14. The electronics package 42 may include a communications module 46, such as Wi-Fi, Bluetooth®, or near field communication, configured for wireless communication with a mobile computing device 48 for controlling the thermoelectric cooler.

The mobile computing device 48 may include an application for sensing and controlling the temperature at the cooling element 12 and the zone of intraoral tenderness. The application may also be configurable by the user for setting a temporal duration of the treatment session via a timer. Data, such as the temperature, duration, date, and user information may be stored in a memory of the mobile computing device 48 as a record of the treatments.

The application may also include a user interface for the user to score the subjective effectiveness of the treatments. The user interface may also allow the user to enter incidents or events that may occur between treatments. Likewise, the user interface may allow the user to enter positioning parameters for each treatment and optionally, a comfort assessment of the hands-free intraoral device 10 and the positioning of the cooling element 12 of the hands-free intraoral device 10 parameters for the treatment session. The data may be analyzed to present one or more trends for the patient's progress or to suggest or recommend one or more positioning parameter adjustments for a subsequent treatment.

The application and the mobile computing device 48 may also communicate the data, with a server, where the data may be retained as a backup for the user. The server may also be configured to analyze data from a plurality of users. An artificial intelligence (AI) module may assess the data to provide insights into treatment and/or device parameters to enhance the effectiveness of the hands-free intraoral device 10 treatment across a broad population. As will be appreciated, the electronics package 42, associated electronics component and application may be utilized with one or more embodiments disclosed herein.

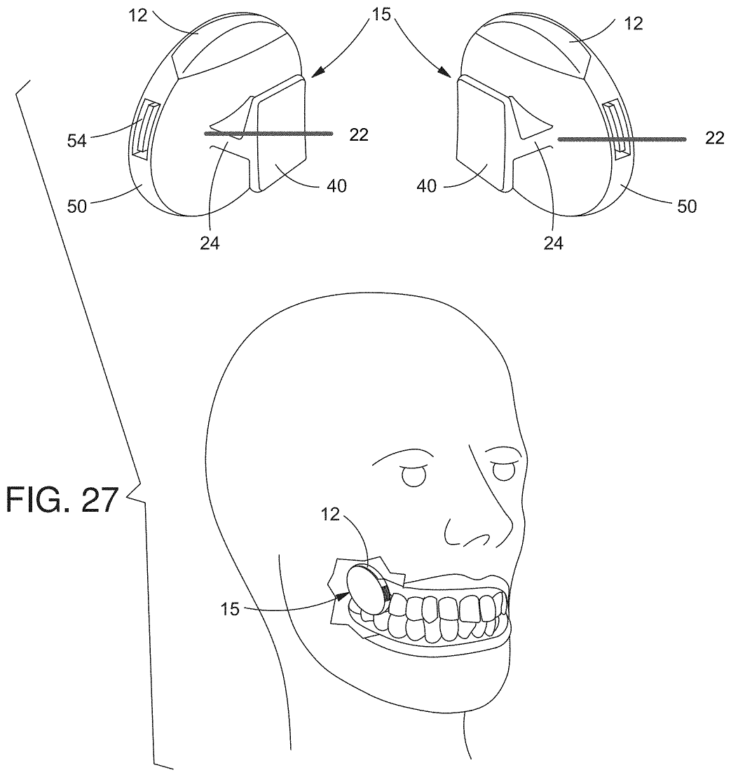

A bite knob embodiment of the hands-free intraoral device 10 is illustrated in reference to FIG. 27. In this embodiment, the carrier includes a disc shaped housing 50 having an inner face and an outer face. The disc shaped housing 50 is configured to contain a TEC, such as the Peltier device. The cooling element 12 is positioned at an upper arch of the disc shaped housing 50, and more preferably extends along an upper margin of the inner face of the disc shaped housing 50 for improved positioning with the zone of intraoral tenderness.

A bite pad 24 extends transversely from the inner face of the disc shaped housing 50 at a connector 22 and is operable by the molars of the user to position the cooling element 12 in abutment with the zone of intraoral tenderness. In this embodiment, a heat exchanger 40 is disposed at an interior end of the bite pad 24. The heat exchanger 40 is configured to communicate heat from the TEC to be dissipated by the vascularization of the user's tongue. The TEC may be powered by a battery source carried within the disc shaped housing 50, which may be charged via a charging port 54. Alternatively, an external power supply may be attached to the charging port 54.

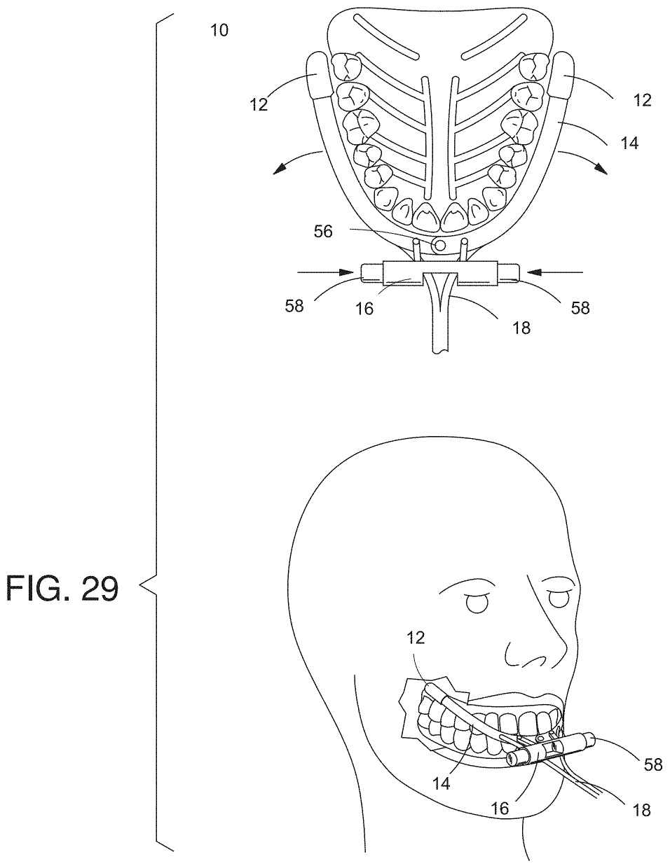

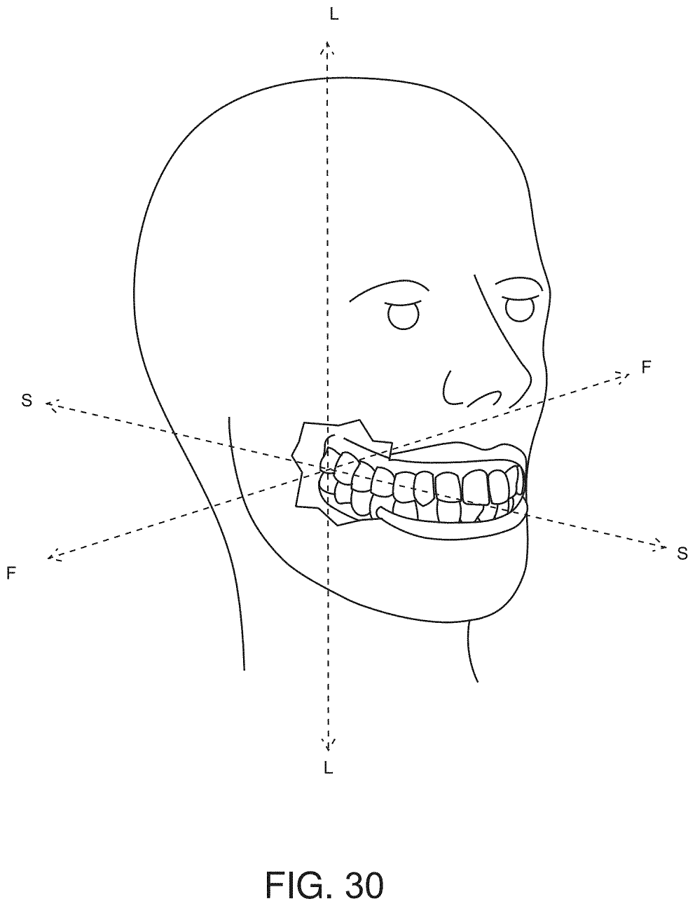

A clamping embodiment of the hands-free intraoral device 10 may be seen in reference to FIG. 29. In this embodiment, the hands-free intraoral device 10 also includes the cooling element 12 disposed at the distal end of the carrier 14. In this embodiment, the left and the right arms of the carrier 14 are releasably biased towards a sagittal plane oriented along a centerline of the intraoral cavity, such that the cooling element 12 is urged in abutment with the zone of intraoral tenderness. The left and the right arms of the carrier 14 may be flexible. A hinge 56 may also couple the left and the right arms of the carrier 14.

In this embodiment, the positioning element 16 includes a tensioning mechanism at a proximal end of the carrier 14, that is operable via a button 58 on one or opposed ends of the positioning element 16. In the embodiment shown, a pressing of the buttons 58 operably urges the left and the right arms of the carrier 14 outwardly, whereas a releasing of the button 58 urges the left and right arms of the carrier 14 inwardly to urge the cooling element 12 in abutment with the zone of intraoral tenderness. In this embodiment, the positioning element 16 may be carried externally of the intraoral cavity.

It should be understood, of course, that the foregoing relates to exemplary embodiments of the invention and that modifications may be made without departing from the spirit and scope of the invention as set forth in the following claims.

1. A hands-free intraoral device for imparting a chilling to a zone of intraoral tenderness within an intraoral cavity of a user, the zone of intraoral tenderness defined by an area of a plexus formed by one or more of a posterior superior and a middle superior alveolar branch of an ipsilateral maxillary nerve, the hands-free intraoral device comprising:
a carrier having a proximal end and a distal end, the distal end configured to be received within the intraoral cavity;
a cooling element disposed at the distal end of the carrier, the cooling element formed of a thermally conductive material selected to impart the chilling to the zone of intraoral tenderness; and
a positioning element position at least a portion of the cooling element in abutment with the zone of intraoral tenderness, wherein the positioning element provides for a hands-free positioning of the cooling element in abutment with the zone of intraoral tenderness.

2. The hands-free intraoral device of claim 1, further comprising:
a connector positioned proximally to the positioning element, the connector acting as a point from which the cooling element and the positioning element separately extend.

3. The hands-free intraoral device of claim 2, the carrier further comprising:
a left prong and a right prong, each of the left prong and the right prong dimensioned to extend the cooling element to contact the zone of intraoral tenderness, when inserted in the intraoral cavity.

4. The hands-free intraoral device of claim 3, the positioning element further comprising:
a bite pad extending the connector towards a sagittal centerline of the intraoral cavity, the bite pad configured to be operably engaged by a biting of one or more molar teeth of a user to position the cooling element in abutment with the zone of intraoral tenderness and to retain the hands-free intraoral device within the intraoral cavity.

5. The hands-free intraoral device of claim 4, wherein the bite pad has a first angular offset about a frontal axis of between about 0° and 25° from the transverse plane.

6. The hands-free intraoral device of claim 4, wherein the bite pad has a first angular offset about a frontal axis of about 5° and 15° from the transverse plane.

7. The hands-free intraoral device of claim 4, wherein the bite pad has a second angular offset about a sagittal axis of between about −15° and +15° from the transverse plane.

8. The hands-free intraoral device of claim 4, wherein the bite pad has a second angular offset of about −10° and +10°.

9. The hands-free intraoral device of claim 4, further comprising:
a guidance plate positioned at an interior and an exterior end of the bite pad.

10. The hands-free intraoral device of claim 9, further comprising:
a tensioning mechanism operable to bias the guidance plate against an interior and an exterior surface of the one or more molar teeth of the user.

11. The hands-free intraoral device of claim 1, the carrier further comprising:
a U-shaped carrier dimensioned to be received within the intraoral cavity, wherein at least a portion of the U-shaped carrier is positioned between an upper jawline of the user and a cheek of the user; and
the cooling element is disposed at a distal end of the U-shaped carrier.

12. The hands-free intraoral device of claim 11, the positioning element further comprising:
a bite pad extending along a distal end of the U-shaped carrier towards a sagittal centerline of the intraoral cavity, the bite pad configured to be operably engaged by a biting of one or more molar teeth of a user position the cooling element in abutment with the zone of intraoral tenderness and to retain the hands-free intraoral device within the intraoral cavity.

13. The hands-free intraoral device of claim 11, further comprising:
a cylindrical extension oriented at an elevated angular offset from the U-shaped carrier about a frontal axis; and
the cooling element disposed at a terminal end of the cylindrical extension.

14. The hands-free intraoral device of claim 1, the positioning element further comprising:
a protrusion defined at the distal end of the carrier; and
the cooling element disposed at a top end of the protrusion.

15. The hands-free intraoral device of claim 1, the positioning element comprising:
means to flexibly urge the cooling element in abutment with the zone of intraoral tenderness.

16. The hands-free intraoral device of claim 1, the positioning element further comprising:
a flexible loop defined at the distal end of the carrier, the flexible loop dimensioned to be resiliently received between an upper jawline, a lower jawline, and a cheek of the user; and
the cooling element is disposed along an upper arc of the flexible loop.

17. The hands-free intraoral device of claim 1, the positioning element further comprising:
at least one flexible arm diverging from the carrier, the at least one flexible arm configured to resiliently engage between a jawline and a cheek of the user to position the cooling element in abutment with the zone of intraoral tenderness.

18. The hands-free intraoral device of claim 1, the positioning element further comprising:
a hook defined at the distal end of the carrier, the hook dimensioned for engagement around a distal end of the maxillary arch of the user; and
the cooling element is positioned at a terminal end of the hook.

19. The hands-free intraoral device of claim 18, wherein the terminal end of the hook is disposed at an elevated angle relative to a transverse plane of the intraoral cavity.

20. The hands-free intraoral device of claim 1, the positioning element further comprising:
a tensioning mechanism at a proximal end of the carrier, the tensioning mechanism biasing a left arm and a right arm of the carrier towards a sagittal plane oriented along a centerline of the intraoral cavity, such that the cooling element is urged in abutment with the zone of intraoral tenderness.

21. A hands-free intraoral device for imparting a heat transfer from a zone of intraoral tenderness within an intraoral cavity of a user, the zone of intraoral tenderness defined by an area of a plexus formed by one or more of a posterior superior and a middle superior alveolar branch of an ipsilateral maxillary nerve, the hands-free intraoral device comprising:
a carrier having a proximal end and a distal end, the distal end configured to be received within the intraoral cavity;
a cooling element disposed at the distal end of the carrier, the cooling element formed of a thermally conductive material selected to impart the heat transfer from the zone of intraoral tenderness to the cooling element;
a heat exchanger configured to transfer heat from the cooling element; and
a positioning element operable to position at least a portion of the cooling element in abutment with the zone of intraoral tenderness, wherein the positioning element provides for a hands-free positioning of the cooling element in abutment with the zone of intraoral tenderness.

22. The hands-free intraoral device of claim 21, the heat exchanger comprising:
a thermoelectric cooler coupled with the cooling element.

23. The hands-free intraoral device of claim 22, further comprising:
an electronics package for control of the thermoelectric cooler is coupled with the carrier.

24. The hands-free intraoral device of claim 23, the electronics package further comprising:
a communications module configured for a wireless communication with a mobile computing device for controlling the thermoelectric cooler.

25. The hands-free intraoral device of claim 1, further comprising:
a conduit to communicate a cooling fluid through the hands-free intraoral device to the cooling element; and
a cooling medium in communication with the conduit, the cooling medium configured to chill the cooling element of the hands-free intraoral device
